(19) United States
(12) Patent Application Publication
(10) Pub. No.: US 2005/0004590 A1
(43) Pub. Date: Jan. 6, 2005

(54) Medical device to remove hubs/ends of intravenous tubing

(76) Inventors: Amneris C. Waters, Hermitage, TN (US); Jeffery Waters, Hermitage, TN (US)

Correspondence Address: LITMAN LAW OFFICES, LTD., P.O. BOX 15035 CRYSTAL CITY STATION, ARLINGTON, VA, 22215, US

(21) Appl. No.: 10/609,488

(22) Filed: Jul. 1, 2003

(51) Int. Cl. A61B 17/32

(52) U.S. Cl. 606/170

(57) ABSTRACT

The medical device to remove the hubs/ends of intravenous tubing is a clamping tool that aids in quick removal and tightening of hubs and other joints in intravenous tubing. The clamping tool has curved jaws which will not break or damage the hubs while tightening or removing connections in IV lines. The clamping tool has a pair of pivotally connected arms having ring handles at one end and gripping jaws at the opposite end. At the end adjacent the ring handles, the arms have opposing ratchets or clamping surfaces which frictionally engage each other when the jaws are approximated to each other, similar to a hemostat. The gripping jaws are relatively wide, having concave openings therein adapted for encasing and gripping about 70-90% of the circumference of the hubs/ends of the intravenous tubing.

### BACKGROUND OF THE INVENTION

1. Field of the Invention

The present invention relates to a medical device for removing and tightening hubs/ends of surgical tubing, and more particularly, to a surgical clamping tool for securing, locking tightening, and removing the hubs/ends of intravenous tubing.

2. Description of the Related Art

For more than twenty years, emergency medical technicians, paramedics and nurses have been using hemostats to remove/tighten the hubs/ends from the medical tubing, a task for which hemostats were never designed. The hemostats tend to strip and damage the outside of the hubs/ends of tubing, and may even break or crack them because too much uneven pressure is applied to remove or tighten the hubs or ends of the tubing. In emergency situations, during the infusion of drugs, if the hubs or tubing ends are damaged, entire lines must be replaced. This is very critical and detrimental to the patient's outcome in the emergency situations.

It is difficult to use one's hands on the hubs and tubing because of the small size of the tubing and the fittings, which are hard to hold and turn while threading and unthreading. Manual manipulation of the hubs and tubing ends also carries with it the risk of contamination. The hubs and tubing ends, being rounded in shape, tend to slide off the jaws of the hemostat due to the relatively small area of contact between the gripping surfaces of the straight hemostat blades and the rounded surface of the hubs or tubing ends. While various devices have been developed for disconnecting tubing, none are as efficacious as the device of the present invention.

U.S. Pat. No. 3,233,313, issued Jul. 22, 1863 to Roth, describes a tube applicator having a pivot structure with a rounded grip for removing the expanded tube. U.S. Pat. No. 4,791,925, issued Dec. 20, 1988 to Mitterer, describes ring removal tool with pivoted spring biased handles for safely removing the ring from the finger.

U.S. Pat. No. 5,913,852, issued Jun. 22, 1999 to Magram, describes a connector for joining medical tubing by deforming the sleeve connecting the tubing. Magram also describes various embodiments of a crimping tool for crimping the sleeve onto tubular sections. The device shown in FIG. 9 of Magram bears a superficial resemblance to the tool of the present invention. However, the tool shown by Magram has thin blade sections which closely approximate each other in order to crimp the sleeve around the tubing, and lacks the wide, inward projecting jaws adapted for gripping a hub disclosed by the present invention. The structure of the Magram tool is incapable of gripping a hub and twisting the hub for removal from an intravenous line without damaging the hub and/or tubing.

U.S. Pat. No. 6,077,280, issued Jun. 20, 2000 to Fossum, describes a surgical clamp for removing foreign bodies, such as, glass, metal and contraceptive capsules through incision during surgical procedures.

None of the above inventions and patents, taken either singly or in combination, is seen to describe the instant invention as claimed. Thus a medical device to remove hubs/ends of intravenous tubing solving the aforementioned problems is desired.

### SUMMARY OF THE INVENTION

The medical device to remove the hubs/ends of intravenous tubing is a clamping tool that aids in quick removal and tightening of hubs and other joints in intravenous tubing. The clamping tool has curved jaws which will not break or damage the hubs while tightening or removing connections in IV lines. The clamping tool has a pair of pivotally connected arms having ring handles at one end and gripping jaws at the opposite end. At the end adjacent the ring handles, the arms have opposing ratchets or clamping surfaces which frictionally engage each other when the jaws are approximated to each other, similar to a hemostat. The gripping jaws are relatively wide, having concave openings therein adapted for encasing and gripping about 70-90% of the circumference of the hubs/ends of the intravenous tubing.

When gripping the hub, the device can be locked into the semicylindrical jaws by tightly locking the clamping surfaces at the finger openings. While the hub is locked into the curved jaws the assembly can be hung temporarily by the ring handles on an IV pole in order to free the user's hands, to safeguard against any contamination resulting from handling of the IV line, and to avoid spillage from or damage to the IV lines.

Accordingly, it is a principal object of the invention to provide a medical device for removing hubs and ends of intravenous tubing having jaws which only extend around between 70-90% of the circumference of a hub, depending upon its size.

It is another object of the invention to provide a medical device for removing hubs or the ends of intravenous tubing which has a plurality of teeth defined on the surfaces of the gripping jaws for better gripping.

It is a further object of the invention to provide a medical device for removing hubs or the ends of intravenous tubing having a pair of pivoting arms with gripping jaws, pivoting arms having ratchets or clamping surfaces for locking the pivoting arms, whereby the device can be used for twisting the hub or tubing without the necessity of maintaining pressure on the pivoting arms.

Still another object of the invention is to provide a medical device for removing the hubs and ends of intravenous tubing which will remain clamped to the hub or tubing end after removal, and which may quickly be hung from an IV support bracket to free the user's hands in an emergency.

It is an object of the invention to provide improved elements and arrangements thereof for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1 is an environmental, perspective view of a medical device to remove hubs/the ends of intravenous tubing according to the present invention.

FIG. 2 is a front view of a medical device to remove hubs/the ends of intravenous tubing according to the present invention.

FIG. 3 is a side view of a medical device to remove hubs/the ends of intravenous tubing according to the present invention.

FIG. 4 is a perspective view of a medical device to remove hubs/the ends of intravenous tubing according to the present invention, illustrating the teeth within the curved gripping jaw.

Similar reference characters denote corresponding features consistently throughout the attached drawings.

### DETAILED DESCRIPTION OF THE PREFERRED EMBODIMENTS

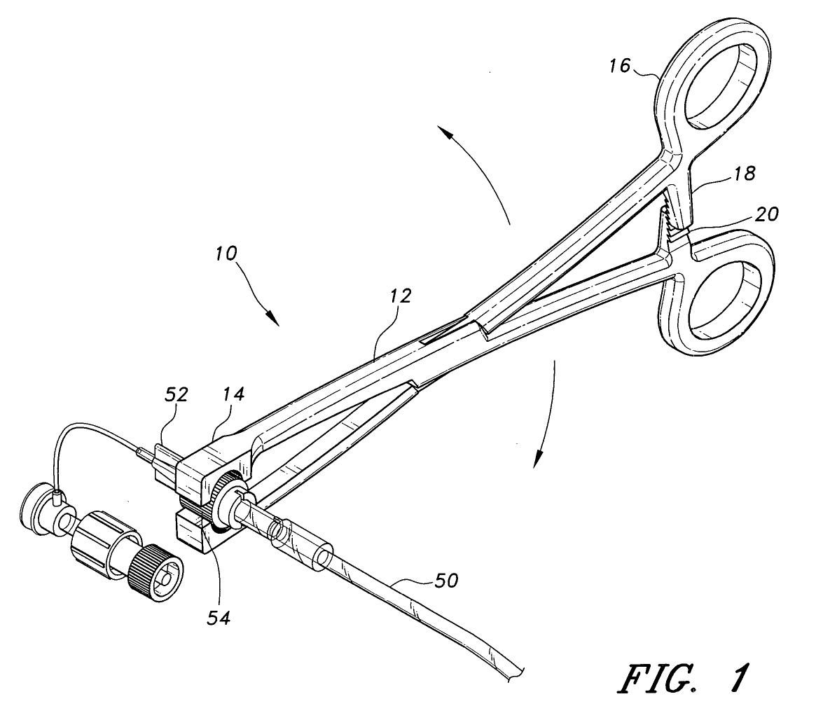

The present invention is a medical device to remove hubs/the ends of intravenous tubing, which essentially comprises a clamping tool, designated generally as 10 in the drawings. As shown in FIG. 1, the clamping tool 10 is designed to remove and tighten hubs 54 on intravenous lines 50 or other medical lines and tubing, such as solution sets, pressure tubing on arterial, central venous lines, Swan-Ganz catheters, extension sets, dead end (red caps), secondary tubing, etc., safely and without damage to the hub 54 or tubing 50 while being used by emergency technicians, paramedics, nurses and other medical staff.

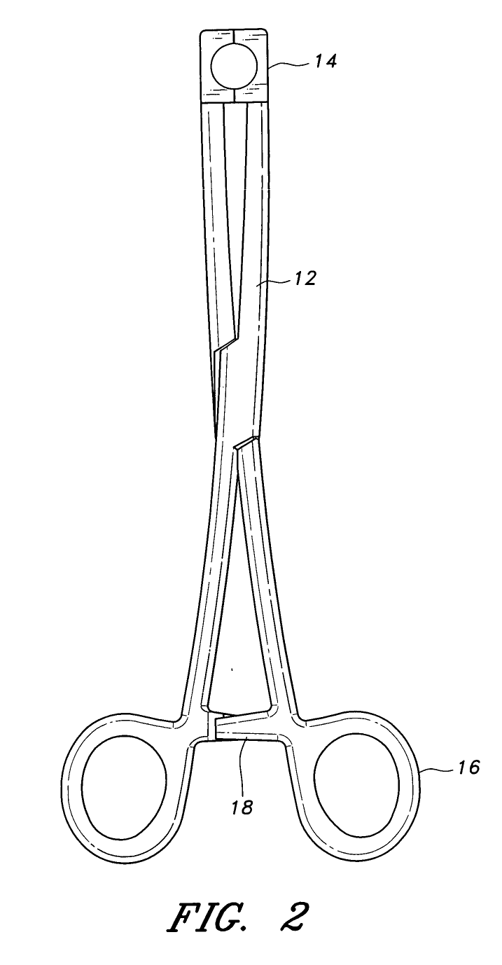

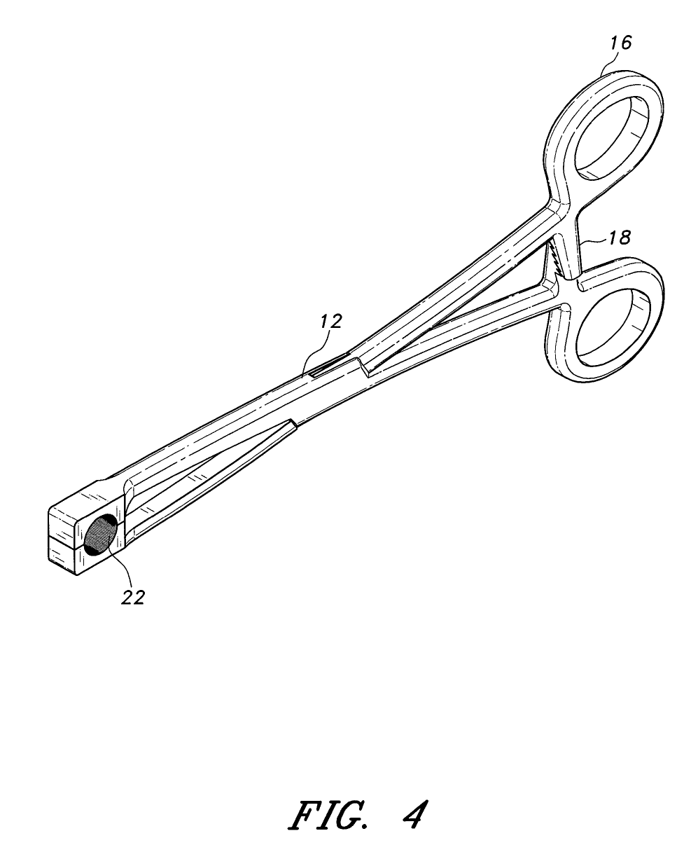

As illustrated in FIGS. 1-4, the clamping tool 10 has a pivotally connected pair of arms 12 having opposing gripping jaws 14 at one end and ring handles 16 at the opposite end. A pair of ratchets 18 having clamping surfaces with ratchet teeth 20 defined thereon extend towards each other adjacent the ring handles 16. The pivotally connected arms 12, ring handles 16, and ratchets 18 closely resemble those of hemostatic forceps, Allis forceps, Babcock forceps, and similar clamping devices.

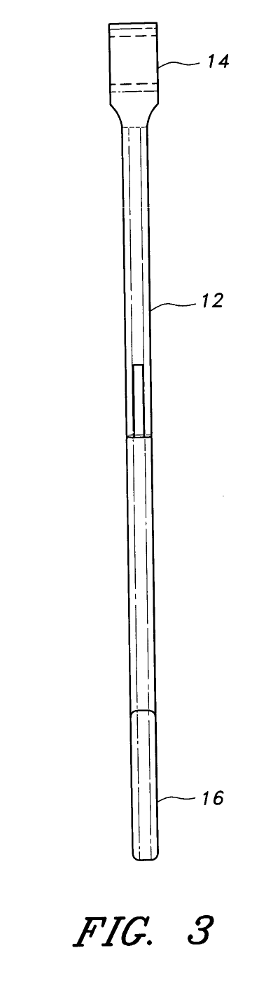

However the clamping tool 10 of the present invention has different gripping jaws 14 from conventional devices which render the present clamping tool 10 particularly well suited for grasping hubs and IV tubing. In particular, the jaws 14 are formed as blocks at the ends of the arms 12 which project towards each other, leaving a gap between the upper portion of the arms 12 when the jaws 14 abut each other. As seen in FIG. 3, each jaw 14 is formed by a block which is thicker than the remainder of the arm 12, each jaw 14 being between ⅜″-⅝″ thick. Each block has a semicylindrical recess defined therein, with upper and lower projections having a planar face, so that when the jaws 14 abut, a cylindrical bore having a diameter of between ⅜″ and one-half inch is formed. This diameter is preferred so that the jaws 14 only extend around between 70-90% of typical IV hubs 54. Each recess has a plurality of gripping teeth 22 defined therein for gripping a hub 54 or tubing end.

In operation, the arms 12 are manipulated by placing the thumb and forefinger through the ring handles 16 and separating the jaws sufficiently to place the jaws around the hub 54 or end of the tubing 50. The ring handles 16 are pressed towards each other, the gripping jaws 14 engaging the hub 54 while simultaneously the ratchet teeth 20 engage one another, incrementally tightening the clamping tool on the hub 54 until the hub 54 is firmly clamped between the jaws 14. Using the fingers, the tool may be twisted to install or remove the hub 54. Since the hub 54 typically caps the IV tubing, the IV line may be hung from an IV support pole by the ring handles 16 to free the user's hands. When finished, the clamping tool 10 is removed from the hub or IV tubing by pushing the ring handles 16 apart to disengage the ratchet teeth 20 while pulling the ring handles 16 away from each other to open the jaws 14.

Preferably the clamping tool 10 is made from stainless steel or other metals typically used in the fabrication of surgical instruments in order to permit cleaning the tool by autoclave.

It is to be understood that the present invention is not limited to the embodiments described above, but encompasses any and all embodiments within the scope of the following claims.

1. A medical device to remove hubs used in intravenous tubing, comprising:
a pair of pivotally connected arms, each arm having a first end and a second end, and a central portion between the first end and the second end, the arms being pivotally connected about midway between the first end and the second end, each arm having a thickness;
a ring formed in the first end of each said arm, the ring defining a ring handle;
a ratchet extending from each arm adjacent the ring handle, each ratchet having a plurality of ratchet teeth defined therein, the ratchets extending towards each other; and
a jaw formed in the second end of each arm, each jaw having a block shape thicker than the central portion and first end of the arm, the jaws projecting from the central portion of the arms towards each other, each jaw having a semicylindrical recess defined therein;
whereby the jaws are adapted for extending around the hub locked in a firm grip by engagement of the ratchets in order to clamp the hub between the jaws.

2. The medical device according to claim 1, wherein each said jaw further comprises a plurality of teeth defined therein adapted for gripping the hub.

3. The medical device according to claim 1, wherein each said jaw is between about ⅜ to ⅝ inches thick.

4. The medical device according to claim 1, wherein each said jaw comprises an upper projection and a lower projection defining the recess, each of the projections having a planar face, the planar faces of the upper and lower projections abutting when said ring handles are drawn together so that the semicylindrical recesses form a cylindrical bore having a diameter between about ⅜ inch and one-half inch.

5. A clamping tool for clamping hubs in intravenous tubing, comprising:
a pair of pivotally connected arms, each arm having a first end and a second end, and a central portion between the first end and the second end, the arms being pivotally connected about midway between the first end and the second end, each arm having a thickness;
a ring formed in the first end of each said arm, the ring defining a ring handle;
a ratchet extending from each arm adjacent the ring handle, each ratchet having a plurality of ratchet teeth defined therein, the ratchets extending towards each other; and
a jaw formed in the second end of each arm, each jaw having each jaw having an upper projection and a lower projection and a semicylindrical recess defined between the upper and lower projections, the jaws projecting from the central portion towards each other;
whereby the jaws are adapted for extending around the hub locked in a firm grip by engagement of the ratchets in order to clamp the hub between the jaws.

6. The clamping tool according to claim 5, wherein each said jaw further comprises a plurality of teeth defined therein adapted for gripping the hub.

7. The clamping tool according to claim 5, wherein each said jaw is between about ⅜ to ⅝ inches thick.

8. The clamping tool according to claim 5, wherein the upper projection and the lower projection of each said jaw has a planar face, the planar faces of the upper and lower projections abutting when said ring handles are drawn together so that the semicylindrical recesses form a cylindrical bore having a diameter between about ⅜ inch and one-half inch.
